(19) United States
(12) Patent Application Publication
(10) Pub. No.: US 2021/0353267 A1
(43) Pub. Date: Nov. 18, 2021

(54) INTERSTITIAL FLUID EXTRACTION

(71) Applicant: Kenota Inc., Kitchener (CA)

(72) Inventors: Yiheng Qin, Mississauga (CA); Christopher J. Harder, Elmira (CA); Dylan Horvath, Toronto (CA); Charlie Man, Toronto (CA); Art De Guzman, Toronto (CA)

(21) Appl. No.: 17/276,218

(22) PCT Filed: Oct. 18, 2019
(86) PCT No.: PCT/CA2019/051480

§ 371 Date: Mar. 15, 2021

Related U.S. Application Data
(60) Provisional application No. 62/748,120, filed on Oct. 19, 2018.

(51) Int. Cl. A61B 10/00

(52) U.S. Cl. CPC A61B 10/0045; A61B 5/14514; A61B 2010/008

(57) ABSTRACT

A device for collection of interstitial fluid that can perform needle assembly actuation, skin piercing, needle assembly repositioning, and secondary skin piercing. Also described is a method for extraction of ISF that results in a significant volume of extracted ISF. The presently described device and method can be used for interstitial fluid extraction and collection in the medical diagnostics field.

### CROSS-REFERENCE TO RELATED APPLICATIONS

This application claims priority to U.S. provisional patent application Ser. No. 62/748,120, filed Oct. 19, 2018, which is hereby incorporated by reference herein in its entirety.

### TECHNICAL FIELD

This invention relates generally to the medical diagnostics field, and more specifically to devices and methods for interstitial fluid (ISF) extraction in the medical diagnostics field.

### BACKGROUND

Interstitial fluid (ISF) surrounds cells and tissues throughout the body and contains serum, water, and other components resulting from extravasation and/or discharge out of blood capillaries and cells. ISF shuttles nutrients and waste products between blood vessels and cells and is a combination of serum and cellular materials. ISF contains sugars, salt, fatty acids, minerals such as calcium, magnesium and potassium, nutrients, amino acids, coenzymes, hormones, neurotransmitters, white blood cells, as well as cellular products and cellular metabolic waste products. ISF can be used for diagnostics as it is a source of unique biomarkers, however it is challenging to sample ISF from the body in sufficient quantities for diagnostic purposes. In one application, ISF has become useful in the monitoring of glucose levels in people with diabetes.

Current devices and methods of use in the medical diagnostics field for use in ISF extraction have several limitations. First, most devices used in diagnostic testing, such as those involved in allergen diagnostics, involve either the collection of blood through a needle and syringe, or through exposure such as in a pinprick test. These methods are not only painful and involved for the user, but especially in cases of exposure, can be potentially harmful. Microneedle devices which perform ISF extraction have been shown to minimize pain and involvement of the user, however their ability to extract large enough volumes of fluid for diagnostic testing is limited.

In one example, U.S. Pat. No. 7,666,150 to Douglas et al. describes a tissue penetration device for blood and interstitial fluid sampling from the skin of a patient having a tissue penetrating member with tissue penetrating tip, and a driver to drive the tip to penetrate the skin concurrently with application of an electric current to the skin to stimulate blood flow.

Current devices and methods for extraction of ISF generally result in collection of very small amounts of ISF, however diagnostic procedures sometimes require larger volumes than can easily be extracted with known devices. Thus, there is a need in the medical diagnostics field for a relatively painless, easy-to-use device and method for interstitial fluid extraction which provides adequate fluid samples for diagnostic testing.

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

An object of the present invention is to provide a device for collection of interstitial fluid that can perform needle assembly actuation, skin piercing, needle assembly repositioning, and secondary skin piercing in a single device. Another object of the present invention is to provide a device and method for extraction of ISF that results in a volume of extracted ISF sufficient for diagnostic testing. Another object of the present invention is to provide an easy to use device for ISF extraction from skin. The presently described device and method can be used for ISF extraction and collection in the medical or direct to consumer diagnostics field.

In an aspect there is provided a device for interstitial fluid extraction comprising: a vacuum chamber having a rim for contacting skin; a plunger extending through the vacuum chamber, the plunger engaging a piercing end with a microneedle array; a vertical translation mechanism coupled to the plunger to extend and retract the plunger; a positioning mechanism coupled to the plunger to position the microneedle array; a vacuum device for creating a negative pressure inside the vacuum chamber; and an actuator to activate the vertical translation mechanism and the positional translation mechanism to cause the plunger to execute a piercing cycle of: extending the piercing end of the plunger toward the rim of the vacuum chamber; retracting the piercing end of the plunger; and repositioning the microneedle array.

In an embodiment of the device, the positioning mechanism rotates the microneedle array.

In another embodiment of the device, the positioning mechanism laterally translates the microneedle array.

In another embodiment of the device, the positioning mechanism laterally translates the microneedle array by imprecision in the positioning mechanism.

In another embodiment, the device further comprises a vacuum device to apply negative pressure to the vacuum chamber.

In another embodiment of the device, the device is configured to execute the piercing cycle a plurality of times upon activation of the actuator.

In another embodiment of the device, the vertical translation mechanism releases kinetic energy to extend the plunger.

In another embodiment, the device further comprises an electrical, electro-mechanical, mechanical, or electronic device to control one or more of the plunger, vertical translation mechanism, positional translation mechanism, vacuum, and actuator.

In another embodiment, the device further comprises an absorbent pad in the vacuum chamber.

In another aspect there is provided a method for extracting interstitial fluid comprising: executing a piercing cycle of: contacting a microneedle array at a location on a skin surface; retracting the microneedle array from the skin surface; and repositioning the microneedle array to a repositioned location on the skin surface; repeating the piercing cycle at least one more time; and applying a negative pressure to the skin surface to extract the interstitial fluid.

In an embodiment, the method further comprises a specific period of time between a last piercing cycle and applying the vacuum to the skin.

In another embodiment of the method, the specific period of time is more than 2 minutes.

In another embodiment, the method further comprises analyzing the fluid for the presence of an analyte.

In another embodiment, the method further comprises repeating the piercing cycle 3, 4, 5, 6, or more than 6 times.

In another embodiment of the method, the microneedle array is rotated at each repositioning step.

In another embodiment of the method, applying the vacuum to the skin further comprises positioning an absorbent pad inside the vacuum chamber for absorbing interstitial fluid while the negative pressure is being applied to the skin.

In another embodiment of the method, applying a vacuum to the skin surface comprises: positioning a vacuum chamber onto the skin surface; applying a vacuum to the vacuum chamber using a vacuum generating device to generate negative pressure inside the vacuum chamber; and releasing the vacuum chamber onto the skin while retaining negative pressure between the vacuum chamber and the skin surface.

In another aspect there is provided a device for interstitial fluid extraction comprising: a vacuum chamber having a rim for contacting a skin surface; a plunger extending through the vacuum chamber, the plunger having a plunger seal to seal with the vacuum chamber; a vertical translation mechanism coupled to the plunger to extend and retract the plunger; a vacuum device for creating a negative pressure inside the vacuum chamber; and an actuator to activate the vertical translation mechanism.

In an embodiment, the device further comprises an absorbent pad inside the vacuum chamber for absorbing interstitial fluid.

In another embodiment, the device further comprises a mechanism for releasably engaging releasing the vacuum chamber onto the skin.

### BRIEF DESCRIPTION OF THE FIGURES

For a better understanding of the present invention, as well as other aspects and further features thereof, reference is made to the following description which is to be used in conjunction with the accompanying drawings, where:

FIG. 1 is a schematic representation of a variation of a device for interstitial fluid extraction;

FIG. 2 is a schematic representation of a variation of a method for interstitial fluid extraction;

FIG. 3 depicts a variation of an applicator and needle assembly for extracting interstitial fluid from a user;

FIG. 4 depicts a variation of a portion of a method for interstitial fluid extraction;

FIG. 5 is a side cross-sectional view of an embodiment of a device for ISF extraction;

FIG. 6 is a front view of the embodiment of the device for ISF extraction shown in FIG. 5;

FIG. 7 is an exploded perspective view of the embodiment of the device for ISF extraction shown in FIG. 5;

FIG. 8 is a top view of the clock spring mechanism of the device for ISF extraction shown in FIG. 5;

FIG. 9a is a front view of another embodiment of a device for ISF extraction;

FIG. 9b is a side cross-sectional view of the embodiment of the device for ISF extraction shown in FIG. 9a;

FIG. 10a is a side cross-sectional view of the embodiment of the device for ISF extraction in FIG. 9a showing the vacuum chamber ejection mechanism;

FIG. 10b is a side cross-sectional view of the embodiment of the device for ISF extraction in FIG. 9a showing the absorbent pad ejection mechanism;

FIG. 11 a is a close-up cross-sectional view of a releasable vacuum cap device comprising a vacuum chamber;

FIG. 11 b is a close-up cross-sectional view of the ISF extraction device shown in FIG. 9b with an engaged vacuum cap device;

FIG. 11c is a is a close-up cross-sectional view of the ISF extraction device shown in FIG. 9b with an engaged vacuum cap device in a first engagement position;

FIG. 11d is a is a close-up cross-sectional view of the ISF extraction device shown in FIG. 9b with an engaged vacuum cap device in a second engagement position;

FIG. 12a is a front view of another embodiment of a device for ISF extraction;

FIG. 12b is a side cross-sectional view of the embodiment of the device for ISF extraction shown in FIG. 12a in a compressed position;

FIG. 12c is a side cross-sectional view of the embodiment of the device for ISF extraction shown in FIG. 12a in an extended position;

FIG. 13a is a front view of a system for ISF extraction;

FIG. 13b is a top perspective view of a diaphragm for ISF extraction;

FIG. 14 is a table of results from Experiments 1 and 2;

FIG. 15 and FIG. 15 ctd. is a table of results from Experiment 3; and

FIG. 16 is a schematic representation of a general embodiment of an ISF extraction device.

### DETAILED DESCRIPTION

The following description of the preferred embodiments of the invention is not intended to limit the invention to these preferred embodiments, but rather to enable any person skilled in the art of to make and use this invention.

Unless defined otherwise, all technical and scientific terms used herein have the same meaning as commonly understood by one of ordinary skill in the art to which this invention belongs.

As used in the specification and claims, the singular forms “a”, “an” and “the” include plural references unless the context clearly dictates otherwise.

The term “comprising” as used herein will be understood to mean that the list following is non-exhaustive and may or may not include any other additional suitable items, for example one or more further feature(s), component(s) and/or element(s) as appropriate.

The term “negative pressure” as used herein, also referred to herein as reduced pressure, refers to a pressure that is less than ambient.

The term “diaphragm” as used herein refers to a concave or cup-like structure having an aperture and at least one deformable surface. The diaphragm can be comprised of a sheet of a semi-flexible material anchored at its periphery and round in shape. Alternatively, the diaphragm can be made of a substantially hard material with at least one deformable surface, wherein deformation of the deformable surface reduces the volume of the diaphragm.

Herein is described a device for collection of interstitial fluid that can perform needle assembly actuation, skin piercing, needle assembly repositioning, and secondary or subsequent skin piercing in a single device. The device can also include features for vacuum application, ISF collection, waste disposal, or a combination thereof, and can be a combination of a reusable device and one or more disposable parts. Controlled rotation and/or repositioning of a needle assembly during interaction with the skin results in a greater number of punctures per surface area and thereby increases volume of extracted ISF from an area of punctured skin. The presently described device can be used for ISF extraction and collection, which is used in the medical diagnostics field. Herein is also described a method for ISF extraction.

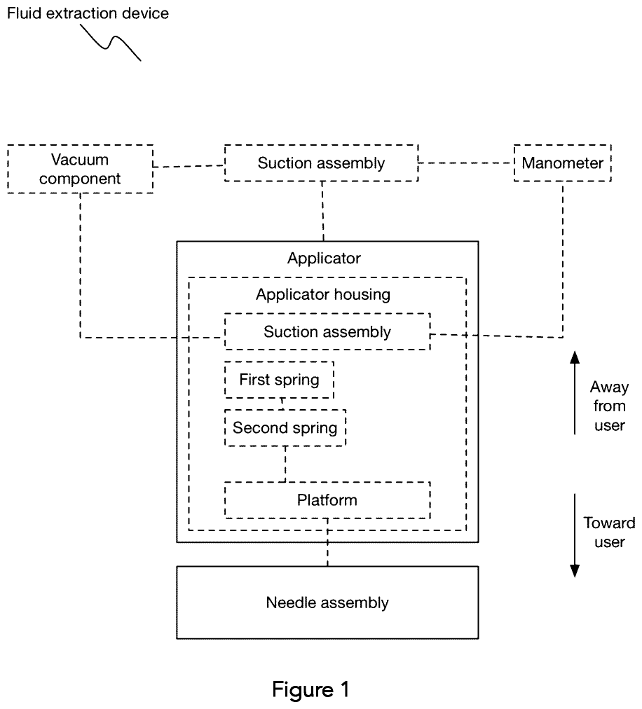

FIG. 1 is a schematic representation of a variation of a device for fluid extraction.

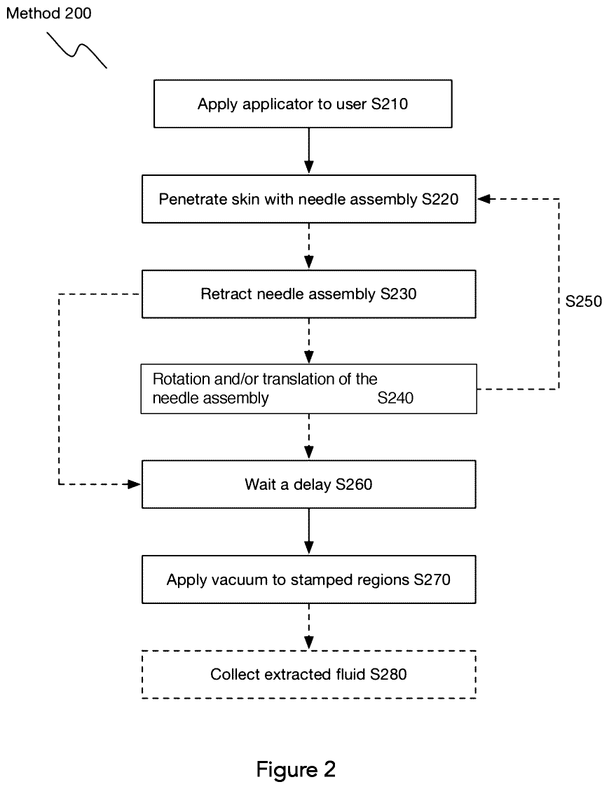

As shown in FIG. 1, an embodiment of a device for ISF extraction includes a needle assembly, an applicator, and a suction assembly. The fluid extraction device can be used to perform the method 200 as described below and in FIG. 2, or to perform any other suitable method for ISF extraction. The needle assembly includes a set of one or more needles, which functions to penetrate skin of the user (e.g., to a depth corresponding to ISF collection, to a depth greater than ISF collection, etc.). Each of the needles is preferably solid but can alternatively have a hollow structure along its entire length, along a partial length, have a slit or groove structure along a needle side wall, or have any other suitable structure. Preferably, each of the needles in the needle assembly is a microneedle (e.g., having a length between 200 and 400 microns, having a length less than 1000 microns, etc.), which is configured to reach a depth of ISF once skin penetration is achieved. Additionally or alternatively, each of the needles can be configured to reach a depth of blood or any other suitable depth corresponding to any fluid composition. Each of the needles in the needle assembly is preferably either pyramidal or cone shaped, but can alternatively have a cylindrical shape (e.g., consistent diameter along length), an otherwise tapered shape (e.g., cylindrical near base of needle and tapered near tip of needle), a slit structure, or any other suitable structure.

The needle assembly preferably includes multiple needles (e.g., microneedles), further preferably an array of needles (e.g., microneedle array), which functions to increase the total number of penetration sites (and thereby the amount of extracted fluid) formed with each penetration. The array preferably includes a regular arrangement (e.g., grid arrangement, equally-spaced arrangement, etc.) of needles, but can alternatively include an irregular array (e.g., unequally-spaced, shaped to conform to a user anatomy, etc.). The pitch of the needle array, equivalently referred to as the needle-to-needle spacing, is preferably between 400 and 600 microns, but can alternatively be less than 400 microns or greater than 600 microns. The array preferably includes a needle array size between 5 by 5 and 25 by 25 needles but can alternatively include any suitable needle array size. The needle array preferably has a needle density between 0.1 and 100 needles per mm² but can alternatively include any suitable needle array density. The needle array preferably includes a flat base at which each needle base is connected (e.g., attached, adhered, formed, etc.), but can alternatively have a curved base (e.g., to conform to user anatomy). The base is preferably a square having a thickness (z-dimension) between 200 and 2000 microns, and a base size (x-y plane) between 50 microns by 50 microns and 150 microns by 150 microns, however the base size can alternatively be smaller than 50 microns by 50 microns or larger than 150 microns by 150 microns. Alternatively, the base can be in the shape of a rectangle, circle, any suitable polygon, or any other shape. The base can further alternatively have any other suitable size and thickness. The needle assembly is generally flat and can have an overall surface area of between 0.1 and 5 cm². The needle assembly is preferably circular, but could also be of any suitable flat shape including, for example, square, hexagonal, or polygonal. The needle assembly can also optionally have a curvature, for example a convex curvature may increase skin penetration near the centre of the needle assembly and a concave curvature may be used to better accommodate vacuum deformation of the skin into a raised dome if the system is under vacuum during skin penetration.

Each of the one or more needles of the needle assembly is preferably made from one or more of medical grade or biocompatible polycarbonate, silicon, an epoxy-based photoresist (e.g., SU-8), polyimide, Poly(methyl methacrylate) (PMMA), Polyether ether ketone (PEEK), liquid crystal polymer (LCP), and stainless steel. Additionally or alternatively, each of the needles can include a combination of any suitable material (e.g., metallic material, non-metallic material) with a medical grade and/or biocompatible material. Any or all of the needles of the needle assembly can have a medical grade or biocompatible coating material such as, for example, parylene-C. In one variation, the needle assembly is in the form of a microneedle array having a set of 300-micron-length microneedles arranged in a regular arrangement having a 0.5-millimeter center-to-center needle spacing.

The applicator functions to initiate movement (e.g., accelerate) of the needle assembly, thereby causing penetration of the skin. The applicator can further function to retract the needle assembly from the user, rotate and/or reposition the needle assembly, and initiate a second skin penetration. The device can further apply vacuum, collect extracted fluid, retain extracted fluid, process extracted fluid, and/or perform any other suitable function. It is noted that rotation or repositioning of the needle assembly by the device or method is accomplished subsequent to retraction of the needle assembly from the skin. Accordingly the skin will be subjected only to an down and up motion perpendicular to the skin for needle puncture. The needle assembly can be rotated and/or translated at each repositioning step between, for example, 1% to 95% of the size of the microneedle array. The applicator preferably includes a housing, which functions to achieve engagement (e.g., contact) between the applicator and the user. In some variations, the housing is also configured to manipulate skin of the user for any or all of the following purposes: achieving better contact between the needle assembly and the user; stretching of the user's skin to increase its tension and thereby increase the ease of needle penetration (e.g., minimize or avoid viscous behavior of skin); increasing a height of skin within the housing (e.g., by creating a domed skin structure); or otherwise improving the application process, retraction process, or overall engagement between the fluid extraction device and the user. In one variation, for instance, a dimension (e.g., diameter) of an end of the housing configured to make contact with the user is chosen (e.g., minimized) to maximize the height of skin (e.g., dome) within that end. The housing can be closed on one end (e.g., end opposing the user), open on both ends, have a pressure-relief vent along a partial or full length of the housing, or have any other suitable configuration. Alternatively, the housing can be formed entirely from the suction chamber described below.

The applicator further preferably includes a platform configured to support (e.g., throughout the method, during application and retraction of the needle assembly, etc.) the needle assembly. The platform preferably includes an attachment mechanism configured to secure the needle assembly to the platform, such as but not limited to: an adhesive (e.g., double-sided tape, glue, partially-cured silicone, fully-cured silicone, etc.) and/or a mechanical attachment site (e.g., set of ridges, indentations, magnet, etc.). The applicator can include any number of actuation mechanisms configured to move (e.g., accelerate) the needle assembly toward skin of the user and away from skin of the user during retraction. In preferred variations, the applicator is a spring-loaded applicator including a set of one or more springs configured to accelerate the needle assembly (and/or platform) toward the user in a first configuration and retract the needle assembly (and/or platform) away from the user in a second configuration. In a specific example, for instance, the applicator is a spring-loaded applicator similar to that in a retractable pen (e.g., having a set of springs, cam body, plunger, stop members, etc.). Alternatively, the needle assembly can be manually accelerated and/or retracted by a user. In one variation, the needle assembly (e.g., microneedle array) is attached (e.g., permanently attached, reversibly attached, temporarily attached, etc.) to a platform, optionally with double-sided tape or other attachment mechanism, and the platform is further connected to a spring configured to accelerate the needle assembly toward a user upon release of a button on the applicator (e.g., a button located at the end of the applicator housing opposing the user). In other preferred embodiments, one or more of the springs and/or one or more of the suction assemblies can comprise, be connected to, or be replaced by one or more electrical, electronic, mechanical, or electromechanical actuators to control the movement of the needle assembly and/or the vacuum device.

The extraction device can further include a suction assembly, which includes any suitable vacuum component (e.g., syringe, vacuum pump, elastic membrane, etc.) and functions to extract fluid from the user. The suction assembly is preferably configured to connect to the applicator housing and achieve fluid communication with the interior of this housing. Additionally or alternatively, the suction assembly can include its own housing separate from the applicator housing, the suction assembly housing configured, for instance, to make contact with the user after the applicator housing has been removed. In one variation, the suction assembly includes a syringe configured to apply either a static or varying pressure (e.g., through a pumping action of the syringe, through a gradual increasing pressure, through a gradually decreasing pressure, etc.) which functions to enhance extraction of fluid from the user. In another variation suction can be provided by movement of a vacuum component, optionally a piston, to provide a reduced pressure environment proximate the skin and site of needle penetration. The suction assembly can additionally or alternatively include any number of components configured to monitor the pressure level, such as a manometer. Additionally or alternatively, the fluid extraction device can include any number and combination of components.

FIG. 2 is a schematic representation of a variation of a method for fluid extraction. As shown in FIG. 2, an embodiment of a method 200 for ISF extraction includes applying an applicator to a user S210, penetrating skin of the user with a needle assembly using the applicator S220, retracting the needle assembly from the user S230, rotating and/or translating the needle assembly relative to the user S240, re-penetrating skin of the user in the same general location as the first penetration with the rotated and/or translated needle assembly using the applicator S250, applying vacuum to the area(s) S270 (e.g., stamped, pierced, or penetrated regions) penetrated by the needle assembly, and collecting ISF S280. Additionally, the method can include any or all of: repeating any or all of the above processes (e.g., penetrating skin of the user with the needle assembly, retracting the needle assembly, rotating the needle assembly, etc.), waiting a delay S260 (e.g., prior to applying vacuum, prior to retracting the needle assembly from the user, etc.), processing the ISF (e.g., to detect one or more antibodies, chemical, or biological markers), or any other suitable step. In one embodiment, the cycle of penetrating the skin of the user, retracting the needle assembly, and rotating and/or translating the needle assembly, also referred to herein as repositioning the needle assembly, can be done multiple times to puncture the skin at various angles and provide a plurality of punctures at substantially the same location on the skin. This method of multiple puncture-retraction-reposition cycles provides a greater number of skin needle penetrations in the same skin area than would be provided with a single penetration and has been found to result in extraction of higher volumes of ISF during the ISF collection step. The puncture-retraction-reposition cycle can be carried out multiple times, manually or automatically by the device. In some embodiments, the number of puncture-retraction-reposition cycles can be done 2, 3, 4, 5, 6, or more than 6 times.

The presently described device and method for extracting ISF can confer several benefits over current fluid extraction devices and methods of use. First, in some variations, the system and method confer the benefit of increased output (e.g., volume, rate of collection, flow rate, percentage by volume, percentage by weight, etc.) of a desired fluid (e.g., ISF) and/or fluid component (e.g., serum, protein, antibody, cells, etc.). In variants, this can be achieved by any or all of: increasing the number and/or density of holes or pierces in the skin prior to desired fluid collection, applying vacuum and/or increasing an amount of applied vacuum, adjusting (e.g., increasing) a needle application force and/or velocity (e.g., to create larger and deeper holes), or through any other suitable process. In some specific examples, for instance, the system and/or method are configured to extract ISF at a high rate (e.g., at least 2 microliters per centimeter squared of skin per minute or greater). Additionally or alternatively, the system and/or method are configured to extract large volumes (e.g., between 5 and 20 microliters, between 20 and 100 microliters, greater than 100 microliters) of ISF in a short time frame (e.g., between 5 and 10 minutes, less than 5 minutes, between 10 and 15 minutes, greater than 15 minutes). Further additionally or alternatively, the device and/or method are configured to extract large volumes of fluid (e.g., ISF) without sacrificing protein content or protein consistency (e.g., as compared to smaller volumes of the same fluid, as compared to similar volumes of blood, etc.). In some specific examples, the overall total protein level of the extracted ISF is lower (e.g., as compared to smaller volumes of ISF, as compared to similar volumes of blood, etc.), while the concentration of one or more antibodies (e.g., Immunoglobulin E [IgE]) is higher, which can confer the added benefit of requiring less volume of extracted fluid than conventional methods of fluid extraction (e.g., blood-based) to collect IgE for antibody testing. Additionally or alternatively, the device and/or method can maximize protein content relative to total fluid sample collected (e.g., protein concentration). In a specific example, for instance, the method can produce 55.7±7.8 microliters of ISF with a protein concentration of 45.6±4.3 milligrams per milliliter. Second, in some variations, the device and method confer the benefit of decreased output (e.g., volume, rate of collection, flow rate, percentage by volume, percentage by weight, etc.) of an undesired fluid (e.g., blood). In a specific example, for instance, the method includes waiting a predetermined amount of time after needle assembly penetration and prior to fluid collection. This allows for healing of the penetrated area and/or blood coagulation, thereby decreasing (or eliminating) the amount of blood present in the collected fluid, such that the majority (or all) of fluid collected is ISF. Third, in some variations, the device and method confer the benefit of improved consistency (e.g., day-to-day consistency, inter-subject consistency, intra-subject consistency, protein-specific consistency) during the fluid extraction process, which can be achieved, for instance, through the repeatable application system and method (number of penetrations, rotations, delay times, etc.). Fourth, in some variations, the device and method confer the benefit of equivalency or near-equivalency between ISF and blood serum collection with respect to any or all of: a sample protein (e.g., antibody, IgE) amount or concentration, total protein amount or concentration, sample volume recovered, or any other suitable parameter(s). Fifth, in some variations, the device and method confer the benefit of recovering cells (e.g., free floating cells, macrophages, etc.) from skin for use in commercial applications such as cell therapy or diagnostics. Additionally or alternatively, the device and method described can confer any other suitable benefit over conventional systems and methods.

A method for extracting fluid from a user can include applying an applicator to a user, penetrating skin of the user with a needle assembly using the applicator, retracting the needle assembly from the user, repositioning the needle assembly relative to the user, re-penetrating skin of the user with a needle assembly using the applicator, retracting the needle assembly from the user, applying vacuum to the area(s) (e.g., stamped regions) penetrated by the needle assembly, and collecting ISF. Additionally, the method can include any or all of: repeating any or all of the above processes (e.g., penetrating skin of the user with the needle assembly, retracting the needle assembly, rotating the needle assembly, etc.), waiting a delay (e.g., prior to applying vacuum, prior to retracting the needle assembly from the user, etc.), processing the ISF (e.g., to detect one or more antibodies), or any other suitable step. The method is preferably performed with a variation of the device described herein, but can alternatively be performed with any suitable device. Various non-limiting embodiments of devices that can be used to perform the method are described below.

The method 200 includes applying an applicator to the user S210, preferably to the skin surface of a forearm region of a user but alternatively to any other skin surface of the user. S210 is preferably performed first in the method 200, but can additionally or alternatively be performed multiple times throughout the method (e.g., prior to each of a set of multiple needle assembly penetration steps) or at any other point during the method. In some variations, S210 can include preparing the skin (e.g., warming or cooling the skin, sanitizing the skin, etc.) for needle assembly penetration. For example, the skin can be warmed to between 20 and 40 degrees Celsius (e.g., using warm water, electrical heater, infrared radiation, friction, and/or other methods); alternatively, the method can be applied to skin at any suitable temperature. In some variations, the applicator can be applied to the user in such a way as to configure the skin for optimal insertion and/or extraction (e.g., stretch skin, form a skin dome through increasing height of skin, etc.). In a specific example, for instance, the applicator is applied to push up the area of skin within the applicator (e.g., within the dimensions of the applicator housing). The method 200 includes penetrating skin with a needle assembly (e.g., microneedle array), which functions to access ISF of the user. S220 is preferably performed with a spring-loaded applicator (such as the one described in the system 100 above), but can alternatively be accelerated manually, with an alternative applicator, inserted with a syringe, or otherwise applied to the user. Penetration of the user's skin with the needle assembly is preferably configured to be smooth and painless, as well as to configured to prevent or minimize potential damage (e.g., fracture, bending, etc.) to the needle structure. In some variations, for instance, an impact force and impact speed are determined based on viscoelastic properties of skin (e.g., based on strain-rate dependence, optimized to enhance elastic properties of skin, optimized to diminish viscous properties of skin, etc.). The needle assembly is preferably applied to the user with an impact force of between 1 and 2 Newtons as the needle assembly reaches the user. Alternatively, the needle assembly can be applied with any suitable impact force (e.g., less than 1 Newton, greater than 2 Newtons, etc.). The needle assembly is further preferably applied with an impact speed between 1.5 and 2.5 m/s just prior to insertion, but alternatively can be applied with any suitable impact speed.

S220 is preferably performed after S210 (e.g., immediately after, in response to, after a predetermined delay, etc.), further preferably multiple times throughout the method (e.g., after S210 and after one or more iterations of S240), but can alternatively be performed a single time throughout the method 200 and/or at any other suitable time(s) during the method 200. The method 200 includes retracting the needle assembly S230, which functions to cease contact between the user and the needle assembly and optionally to permit fluid to flow to an external skin surface of the user for future collection. The needle assembly is preferably retracted using an applicator (e.g., same applicator as used for needle assembly penetration, a different applicator as that used for needle assembly penetration, an applicator as described above, spring-loaded applicator, etc.), but can alternatively be retracted manually. The needle assembly can be retracted at the same speed as insertion speed, faster (e.g., to overcome sticking or suction with the user), or slower (e.g., to prevent detachment of the needle assembly from an applicator platform). Additionally or alternatively, the speed of retraction can be determined based on the viscoelastic properties of skin (e.g., increase viscous properties, increase elastic properties, etc.). As a result of S230, the needle assembly is preferably fully retracted (e.g., the needle is fully retracted from the skin, the needle assembly is displaced to a distance at least equal to the length of the needle(s), etc.), but can alternatively be partially retracted (e.g., displaced to a distance less than the length of the needle(s)) or otherwise retracted. S230 is performed after S220, and can be immediately performed, performed after a predetermined delay has passed (e.g., delay less than 10 seconds, greater than 10 seconds, less than 1 minute, less than 5 minutes, etc.), or performed after a random or variable delay has passed. S230 is preferably performed multiple times throughout the method 200 (e.g., after each of a set of multiple instances of S220), a single time during the method 200, or at any other suitable time(s). The method 200 preferably includes repositioning, for example translating and/or rotating, the needle assembly relative to the user S240, which functions to increase the volume of fluid (e.g., ISF) extracted from the user (e.g., during a fixed period of time) and/or decrease the time required to extract a fixed amount of fluid by increasing the number of penetration sites (e.g., puncture sides, surface area of punctured skin, etc.) from which fluid is extracted. Alternatively, the needle assembly can maintain a static position relative to the user or the user can move relative to the needle assembly.

The needle assembly is preferably rotated and/or translated by a predetermined amount, which can vary widely depending on the configuration of the device. In some uses the needle assembly can be rotated by between, for example, 1 and 60 degrees, or between 1 and 90 degrees, but can alternatively rotate by a predetermined amount less than 60 degrees (e.g., by 45 degrees or less), greater than 90 degrees, a variable amount, a random amount, an amount determined by user, an amount more than or less than the separation distance between adjacent needles in the array, or any suitable amount. For controlled rotation, the needle assembly is preferably rotated about a central axis of the needle assembly (e.g., rotates about a single point on the user), but can alternatively be rotated about an axis offset from the needle assembly center, laterally shifted, or otherwise moved. In some variations, the amount of rotation is configured to prevent needle insertion at the same location twice (e.g., either the same needle or a different needle), which can function to prevent interruption of healing (e.g., coagulation of blood), minimize amount of bleeding, or perform any other suitable function. In some variations, the applicator has an indicator (e.g., tab, button on side of applicator as opposed to or in addition to a symmetrically-oriented button on top of applicator, etc.) to assist in proper rotation (e.g., during manual rotation). Additionally or alternatively, the applicator can be configured for automatic rotation (e.g., a ratcheting system, with a motor in the applicator, etc.). Additionally or alternatively, S240 can include translation of the needle assembly (e.g., to a distance other than the needle spacing of a microneedle array, to a distance greater than the needle spacing of a microneedle array, to distance less than the spacing of a microneedle array, etc.) relative to the user, which can similarly function to increase the volume of fluid extracted (e.g., during a fixed time period) and/or decrease the time required to extract a fixed amount of fluid by increasing the density of penetrations in unit skin surface area. In some variations, such as those involving manual positioning by a user, both rotation and translation of the needle assembly occurs. Alternatively, the translation mechanism can laterally translate the microneedle array by imprecision in the positional translation mechanism. This translation (equivalently referred to as a shift) can be in one or both of x and y directions, wherein skin of the user forms the x-y plane. In a specific example, a shift distance (e.g., average shift distance, predetermined shift distance, etc.) of between 1 micron and 3 millimeters can be applied between stampings or piercings. Alternatively, any other suitable shift distance can be applied.

S240 is preferably performed after S230, further preferably after all but the last time of the multiple iterations of S230, but can alternatively be performed after each iteration of S230 (e.g., to return the needle assembly to a set beginning orientation), or any number of times during any part of the method 200. In variations in which S240 is performed multiple times, the amount of rotation and/or translation is preferably the same for each time S240 is performed, but can alternatively be variable (e.g., increasing amount, decreasing amount, alternating amount, random amount, etc.). The method preferably includes repeating S220, S230, and S240, but can additionally or alternatively include repeating any of the other steps (e.g., S210, S260, S270, S280, etc.) of the method 200, performing each step a single time, or otherwise performing the method. For each repetition, the ordering of steps from S220 to S230 to S240 is preferably maintained but can alternatively be altered. As described previously, S220 and S230 can be performed the same number of times while S240 is performed a fewer number of times (e.g., one less time). Alternatively, S240 can be performed the same number of times as S220 and/or S230, S230 can be performed a different number of times than S220, or the steps can be otherwise performed. In one variation, each of the repeated method steps is preferably performed for a total of between two and seven times. In some variations, for instance, the needle assembly penetrates the skin and is subsequently retracted for a total of five times, with rotations and/or translations of the needle assembly performed in-between. However, any suitable number of iterations of the aforementioned processes can be performed. In variations of the method including repeated steps, preferably the same needle assembly and applicator are used in each step; alternatively, different components can be used (e.g., replaced after a predetermined number of uses). The repeated steps are preferably performed in rapid succession (e.g., less than 1 second between a retraction and subsequent needle penetration), but can alternatively be performed with any suitable timing or frequency (e.g., greater than 1 second between steps). Although the method described herein is exemplified using a set of springs, it is understood that the same method can be carried out by using one or more electrical, electronic, or electromechanical actuators to control the movement of the needle assembly and/or the vacuum device.

The method 200 preferably includes waiting a delay S260 prior to S270, described below, which functions to allow time for blood to coagulate and therefore minimize the amount and/or percentage of blood that is extracted from the user. The delay is preferably between 2 and 15 minutes, further preferably between 2 and 5 minutes, but can additionally or alternatively last for any suitable duration of time (e.g., less than 2 minutes, greater than 15 minutes, etc.), a duration of time determined by the user, a duration of time determined based on a parameter (e.g., total volume collected, total protein collected, etc.). S260 is preferably performed between S250 and S270 but can additionally or alternatively be performed between iterations of S250 (delay between stampings), prior to any step of the method 200, or after any step of the method 200. In some variations, a delay between 3 and 15 minutes is waited after the final iteration of S240 and prior to S270.

The method preferably includes applying vacuum (e.g. reduced pressure or suction) proximal to (e.g., over, in contact with, at an offset from) the stamped regions (e.g., areas having been penetrated or pierced by the needle assembly) on the user's skin S270, which functions to extract fluid (e.g., ISF) from the user, thereby creating a puddle of this fluid on a skin surface of the user. Vacuum is preferably applied with a suction assembly. In an embodiment the suction assembly can be a syringe adjusted to 6 pounds per square inch (psi), which can apply a continuous or varying (e.g., through a pumping action) pressure. The suction assembly preferably covers all or a majority of the penetrated areas of the user's skin, but can alternatively cover a subset of the penetrated areas. Alternatively, a pressure differential can be applied with any suitable component or process. In some instances, it has been observed that the amount of vacuum (magnitude of pressure differential) has been observed to affect specific and/or total protein concentrations of extracted fluid; the amount and duration can be selected to account for this (e.g., maintain a specified protein concentration). The pressure applied in S270 is preferably between 3 and 9 psi (e.g., 6 psi) at an application rate between 0.2 and 3 psi/s, with an air leak rate less than 0.1 psi/min; alternatively, vacuum can be applied with any suitable parameters. The duration for which vacuum is applied (extraction time) is preferably between 5 and 60 minutes, further preferably between 5 and 15 minutes, but can alternatively be for any suitable amount of time. S270 is preferably performed after S260 but can additionally or alternatively be performed in place of S260, between S230 and S240, multiple times throughout the method, or at any other suitable time.

The method 200 can optionally include collecting extracted fluid S280 after S270 has ceased. At this point, the fluid (e.g., ISF) has pooled at the skin surface (e.g., naturally or due to the vacuum applied during S270) and can be collected with any suitable fluid collection mechanism (e.g., capillary tube, wick, scraper, absorbent pad, etc.) or process (e.g., capillary force). In some variations, S280 can include decreasing the pressure applied in S270 by any suitable amount and at any suitable rate (e.g., gradually decreasing the pressure to a final value prior to removing the suction assembly). S280 can be performed during or after S270. In the former instance, the collection mechanism is preferably integrated or built into the suction assembly (e.g., the suction assembly includes a capillary tube fluidly connected to the suction lumen, etc.), but can be otherwise configured. In the latter instance, S280 can be performed immediately after S270 is completed, after a predetermined delay (e.g., more than 10 seconds, more than 1 minute, less than 10 minutes, more than 10 minutes, etc.), or at any suitable time.

In one specific example of the method, the needle assembly is formed from medical grade polycarbonate microneedles arranged in a 10 needle by 10 needle array. Each of the microneedles is pyramidal having a 300-micron needle height and 500-micron needle pitch. The microneedles are arranged on an 8-millimiter×8-millimeter×1-millimeter base for stamping. The applicator provides an impact force of 1.6 Newtons and a landing velocity of 2 meters per second. A 5 minute delay is executed prior to applying vacuum at a value of 6 psi, a speed of 0.6 psi per second for a duration of 15 minutes.

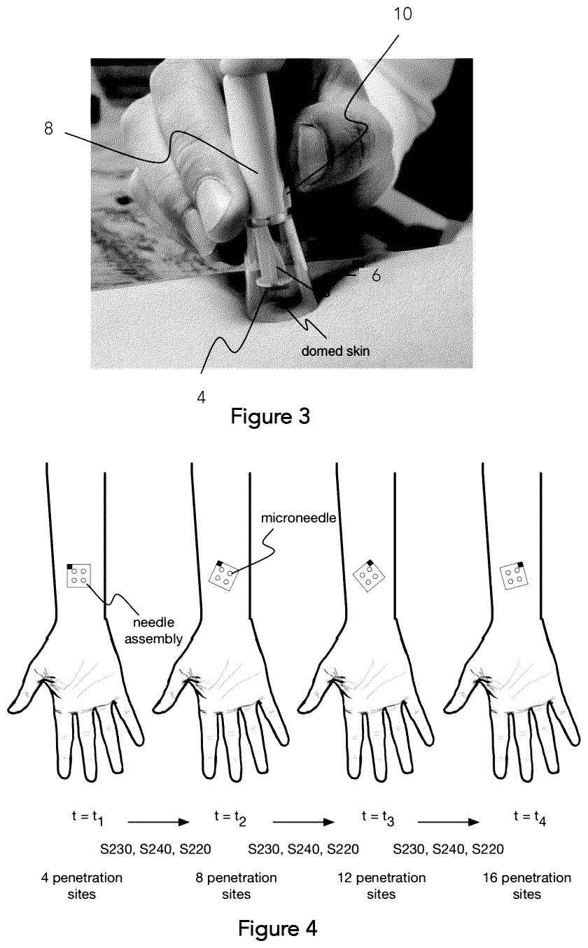

FIG. 3 depicts a variation of an applicator and needle assembly for extracting interstitial fluid from a user. An applicator with device housing 8 is shown applied to skin. Plunger 6 is attached to a needle assembly 4 configured to make contact with the skin when the plunger 6 is depressed. Actuator 10, shown here as a button, activates a spring-loaded applicator to pierce the skin with the needle assembly 4. The skin under the device housing 8 shown is domed resulting from downward pressure of the device housing 8 on the skin. Domed skin under the device can also optionally be achieved by providing negative pressure in the area of skin above the applicator.

FIG. 4 depicts a variation of a portion of a method for interstitial fluid extraction. As shown, skin penetration occurs when the needle assembly is applied to the skin, and the needle assembly is rotated between each skin penetration to provide different sites of penetration in the same general area on the skin. Shown in FIG. 4 are four penetration sites and four different rotations and needle penetrations of the skin according to the method described above and in FIG. 2. Each iteration of the penetration-reposition cycle provides approximately another n skin penetrations, where n is the number of microneedles in the needle assembly.

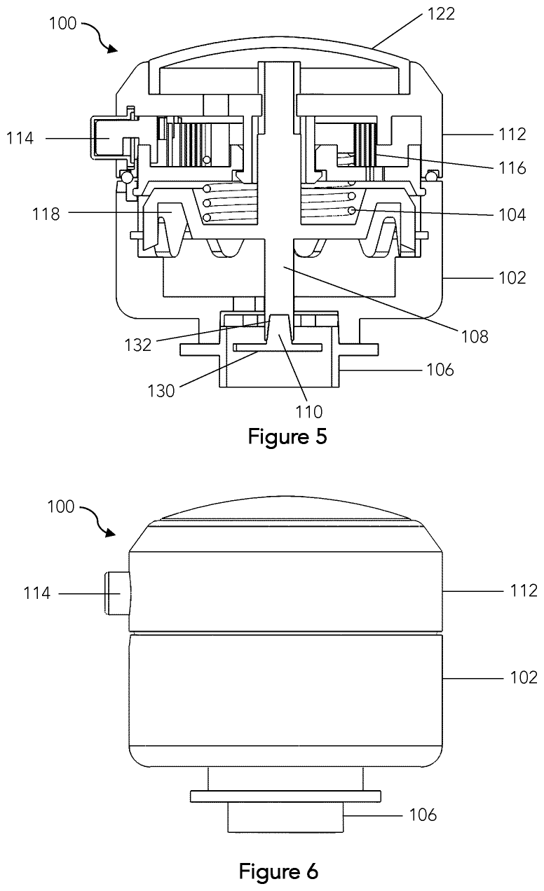

FIG. 5 is a side cross-sectional view of another embodiment of a device 100 for ISF extraction. ISF extraction device 100 has a device housing 102 releasably connected to a vacuum chamber 106. The ISF extraction device 100 is preferably a reusable device and the vacuum chamber 106 with needle assembly 110 is preferably a disposable part. This device is an all-in device that is attached to the microneedle array 130 and can perform microneedle grabbing, skin piercing, microneedle repositioning, vacuum application, ISF collection, and waste disposal. The needle assembly 110 can comprise a bare microneedle array 130 without a coating or covering, or the microneedle array 130 can optionally be coated or covered with one or more layers. In one example an absorbent pad can be placed adjacent to and/or on top of the microneedle array 130 for ISF collection, optionally when a negative pressure with respect to ambient pressure is being applied inside the vacuum chamber 106. The absorbent pad can have a thickness of, for example, 0.01-5.0 mm, and can be placed either around the microneedle array 130, on top of or covering the microneedle array 130, or a combination thereof. The absorbent pad can be, for example, a delicate task wipe, cotton fibre material, nitrocellulose membrane, or other membrane material that can absorb ISF. The absorbent pad can also comprise more than one absorbent pad of the same or different material. The needle assembly can also comprise more than one or multiple absorbent pads of any suitable size and shape.

The plunger 108 on the vacuum chamber 106 acts as a tip to mechanically and reversibly couple with the needle assembly 110, preferably through a press-fit mechanism. The needle assembly 110 has a piercing end with microneedle array 130. The rim on the vacuum chamber 106 and the skin surface can form an air-tight seal for vacuum application subsequent to skin piercing with the needle assembly 110. The vacuum chamber 106 can also be released or ejected from the device housing 102 subsequent to skin piercing to retain the vacuum on the skin to extract the ISF without the additional weight of the ISF extraction device mechanical components. To release the vacuum chamber 106 from the device housing 102, the vacuum chamber is held down on the skin and the device housing 102 is lifted. The plunger 108 on the vacuum chamber 106 and the connected needle assembly 110 is also lifted until the frustoconical piston 132 on the top of the needle assembly 110 is wedged into an aperture in the top of vacuum chamber 106. The further lifting of the plunger 108 disconnects the piston 132 on the needle assembly 110, resulting in the vacuum chamber 106 and the needle assembly 110 being released or ejected from the device housing 102. The vertical translation mechanism provided by the combination of the plunger 108 and coil spring 104 uses the kinetic energy stored in the coil spring 104 to move the needle assembly 110 down toward the skin to effect piercing by the microneedle array 130. The piston 132 on the needle assembly 110, the vacuum chamber 106, and the skin form an enclosure with negative pressure in it to provide a conducive vacuum environment to extract ISF from the skin. The button 114 on the ISF extraction device 100 allows the user to hold the device and activate the skin penetration cycle. The button 114 triggers the stamping and rotation of the needle assembly 110 and microneedle array 130.

The plunge mechanism for the needle assembly 110 has a rod which forms a rigid connection to the loaded the needle assembly 110 to provide a vertical translation mechanism for the microneedle array 130. The plunge mechanism also comprises a plunge gear 118 having one or more teeth facing in the direction of the needle assembly 110 to act as a rotational positioning mechanism to rotationally position and reposition the microneedle array 130. The teeth in the plunge gear 118 mate with teeth of a fixed gear or any other type of guiding structure inside the device housing 102 capable of mating with the plunge gear teeth so that when the plunge gear 118 rotates, its rotation is guided by the teeth inside the device housing 102, causing the plunge gear 118 to travel up and down in the vertical direction toward and away from the vacuum chamber 106. Due to the rigid connection between the piston 132 on the needle assembly 110 and the plunger 108 on the plunge gear 118 in the vacuum chamber, the up/down travel and rotation of the needle assembly 110 through the positioning mechanism are realized. During upward travel, the plunge gear 118 is pushed up by the teeth profile in the device housing 102, and the coil spring 104 positioned on top of the plunge gear 118 is compressed. When the top of the teeth on the plunge gear 118 are pushed against the top of the teeth inside the device housing 102 due to the compression from the coil spring 104, the width of the gear teeth on the plunge gear 118 align inside the teeth of the device housing 102. This determines the interval between two sequential stamping or penetration events. Once the top of the gears on the plunge gear 118 meets the spacing between the teeth on the gear inside the device housing 102 the plunge gear 118 starts to travel downwards until it hits the gear root surface, providing both a vertical and rotational mechanism. The width of the gear surface inside the device housing 102 determines the duration the microneedle array staying on the skin. The distance between the gear root surface and the top of each tooth in the gear mechanism defines the maximum up and down travel distance of the needle assembly 110 relative to the bottom of vacuum chamber 106 where the vacuum chamber 106 interfaces with the skin. This distance and the spring constant of the coil spring 104 define the impacting speed and force of the needle assembly 110 onto the skin. Although the device described herein is exemplified using a set of springs, it is understood that the same method can be carried out by using one or more alternative or additional hydraulic, electrical, electronic, or electromechanical actuators to control the movement of the needle assembly, vacuum chamber, microneedle array, and/or the vacuum device.

The number of stamps or penetrations applied to the skin by the needle assembly 110 is determined by the number of teeth on the plunge gear 118 that rotate pass a starting reference point, which is dependent on the force stored by clock spring 116 which provides the applied force for the rotation of the plunge gear 118 relative to the device housing 102. The rotation angle of the microneedle array is defined by the pitch of the teeth on plunge gear 118. This rotation-plunge mechanism which serves as a rotational positioning mechanism and a vertical translation mechanism, is driven by a housing twist top 112, a clock spring 116, and a leaf spring. When the housing twist top 112 is pre-rotated and fixed, the clock spring 116 is loaded and ready for deployment. The button 114 on the housing twist top 112 releases the clock spring 116 from its pre-rotated position with the movement of a leaf spring, and the clock spring 116 is released to drive the rotation of the plunge gear 118. The pre-rotation amount of the housing twist top 112 defines the number stamps, vertical translations, or penetrations the device will execute of the needle assembly 110 onto the skin. On top of the housing twist top 112, an elastic vacuum membrane 122 acts as a vacuum device and is used to create a vacuum environment inside the device and vacuum membrane 122 when the vacuum membrane 122 is pressed down and then released. All interfaces between parts are sealed so that a vacuum environment can be formed inside the device when pressed onto the skin.

Another mechanism that can be used as a rotational positioning mechanism to rotate the microneedle array is a bi-stable cam system actuated, for example, by a guide pin and a compression spring. In the microneedle penetration/stamping position, the spring is pre-loaded in order to keep the upper and lower cams pressed together. The needle assembly is attached to the upper cam. When the needle assembly is loaded in the launching position, the spring is pressed to the change point, and the upper cam moves up relative to the lower cam and clears the guide pin. The force of the compressed spring is larger than the friction on the inclined surfaces of the cams, and the upper cam rotates relative to the lower cam as the upper cam is lifted to the launching position. By compressing the spring past the change point again, the lower and upper cam rotates relatively, and the lower cam travels upwards relative to the upper cam. Since the position and rotation of the lower cam is fixed, the upper cam acts as a vertical translation mechanism and is driven downwards back to the needle assembly penetration/stamping position. The impacting force and speed of the needle assembly onto the skin is determined by the spring constant and the upper cam travel distance after it passes the change point the second time.

FIG. 6 is a front view of the embodiment of a device for ISF extraction shown in FIG. 5. ISF extraction device 100 has a device housing 102 in sealing engagement with vacuum chamber 106. Housing twist top 112 is rotatable about device housing 102 to wind and activate a clock spring inside the ISF extraction device 100. Actuator button 114 is attached to a leaf spring which releases the clock spring to activate the penetration-retraction-rotation cycles of the ISF extraction device 100.

FIG. 7 is an exploded view of the embodiment of a device for ISF extraction shown in FIG. 5. From left to right, needle assembly 110 fits inside and is mechanically engaged with vacuum chamber 106. A plunger engaged with a needle assembly 110 which acts as a piercing end enables transfer of force from coil spring 104 inside device housing 102 to actuate the puncture-retraction-rotation cycle of the ISF extraction device. Plunge gear 118 is engaged with device housing 102 with an intermediate O-ring gasket 124 to provide sealing between the elements. In the housing twist top 112 clock spring 116 is positioned on and movably engaged with twist clip 126. Button 114 is mechanically engaged with leaf spring 120 to actuate clock spring 116. Vacuum membrane 122 positioned at the top of housing twist top 112 is capable of creating a vacuum to the device and vacuum chamber 106 sufficient to extract ISF from skin subsequent at least one puncture-retraction-rotation cycle effected by the ISF extraction device.

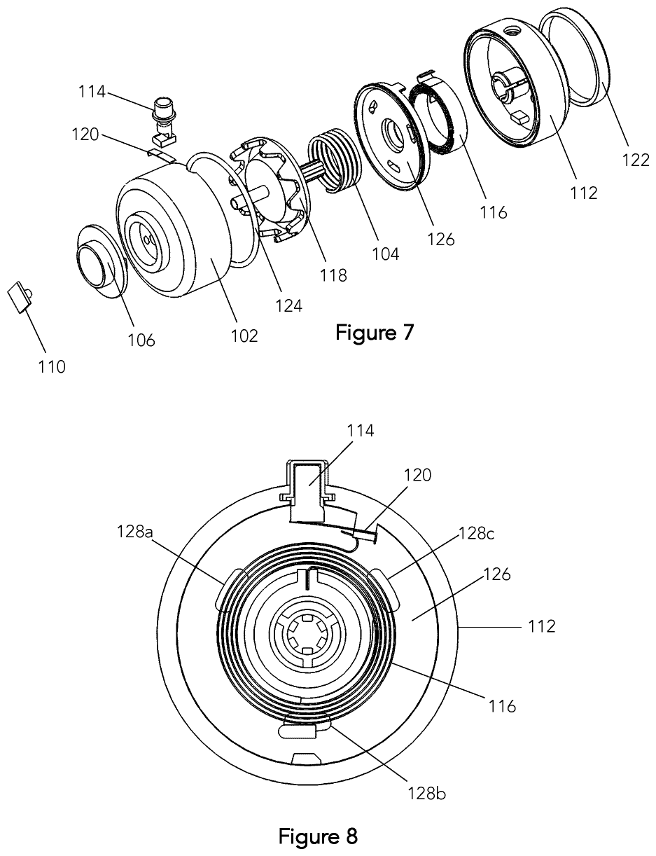

FIG. 8 is a top view of the clock spring mechanism of the device for ISF extraction device shown in FIG. 5. Inside housing twist top 112 clock spring 116 is held in place by twist clip 126 having spring retention elements 128a, 128b, 128c. Button 114 is in mechanical engagement or contact with leaf spring 120, and release of leaf spring 120 upon actuation of button 114 activates clock spring 116 to execute the puncture-retraction-rotation of the ISF extraction device.

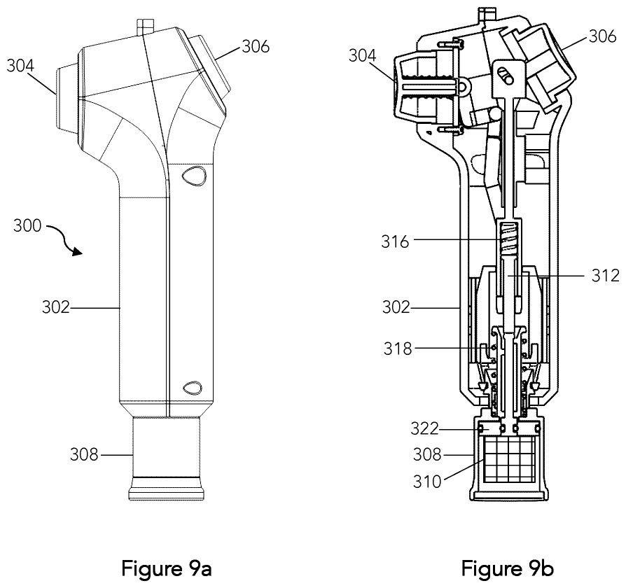

FIG. 9a is a front view of another embodiment of a device for ISF extraction shown with index trigger 304 and thumb trigger 306. This ISF extraction device comprising device housing 302 can be used to pick up a vacuum chamber 308, load the vacuum chamber 308 onto skin, trigger the vacuum to extract ISF from the skin, collect the ISF, remove the vacuum chamber 308 from skin, and dispose of the vacuum chamber 308. The lower part of the ISF extraction device 300 is a vacuum chamber 308 or container that can be press-fit into the device to create a vacuum seal between the device and the skin. Optionally, the vacuum chamber 308 can also be fitted with an absorbent pad to collect ISF. The absorbent pad can be positioned in the vacuum chamber 308, with optional adhesive on the needle assembly to adhere to the absorbent pad. Preferably the adhesive is not on the center of the plunger tip. Optionally a flexible frame can be added between the plunger and the pad to hold flexible and fragile membranes and/or absorbent pads.

FIG. 9b is a side cross-sectional view of the embodiment of the device for ISF extraction shown in FIG. 9a. Device housing 302 for the ISF extraction device has an index trigger 304 for operating a mechanism to expel the vacuum cap from the device, and thumb trigger 306 for operating a plunger 312 to expel absorbent pad 310 from the vacuum chamber 308. First spring 316 in the ISF extraction device is biased in a downward direction and acts to put pressure on plunger 312 to expel absorbent pad 310 from the vacuum chamber 308. Second spring 318 integrated with vacuum chamber 308. Absorbent pad 310 is shown adhered to plunger seal 322. During placement of the vacuum chamber 308 onto a skin surface for extraction for ISF, it is understood that the plunger seal 322 will be in a lowered position such that absorbent pad 310 is either in contact with skin or positioned just above the skin so that it can absorb ISF.

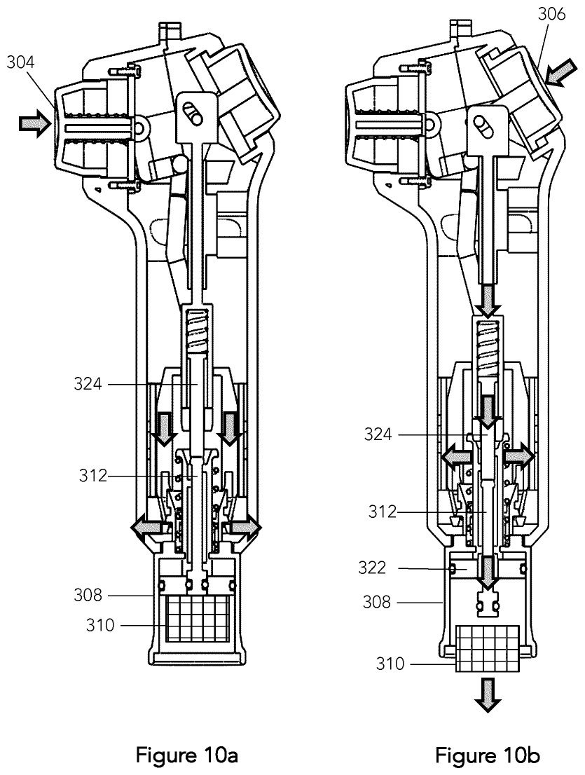

FIG. 10a is a side cross-sectional view of the embodiment of the device for ISF extraction in FIG. 9 showing the vacuum chamber ejection mechanism. In use, the ISF extraction device with vacuum chamber 308 is pushed onto skin. If index trigger 304 is pressed, piston rod 324 exerts a downward force onto plunger 312 into vacuum chamber 308 in the direction indicated by the arrows, detaching and ejecting the vacuum chamber 308 from the device. If the vacuum chamber 308 is ejected when under negative pressure on skin, the vacuum chamber 308 can remain on skin and continue to provide a negative pressure environment adjacent to pierced skin to draw out ISF from the skin. Ejection of the vacuum chamber 308 after use as waste can also be done using the same process.

FIG. 10b is a side cross-sectional view of the embodiment of the device for ISF extraction in FIG. 9a showing the absorbent pad ejection mechanism. Upon activation of thumb trigger 306, piston rod 324 is pushed downward to expel the absorbent pad 310 from the vacuum chamber 308. In one embodiment, the thumb trigger 306 on the ISF extraction device can be pressed to trigger a series of lever arms to press down the piston rod 324, which transfers force to the plunger 312 inside the vacuum cap device. The piston rod 324 and plunger 312 travel downwards and pushes the attached absorbent pad 310 so that the pad is detached from the plunger seal 322. This action disposes the absorbent pad 310 with collected ISF. The index trigger 304 can be again pressed to trigger a series of lever arms. The lever arms can expand the top part of the vacuum cap device and release the connections between the vacuum cap and the device to enable the vacuum cap device to be detached from the ISF device and disposed of. Once the ISF extraction and collection is completed, the ISF extraction device can be pushed onto a new vacuum cap device.

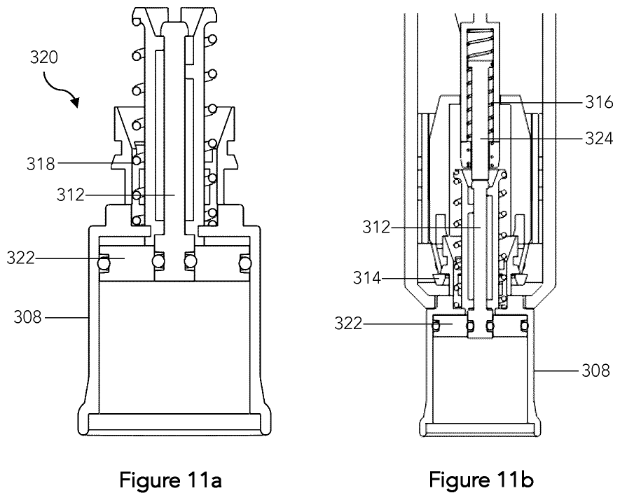

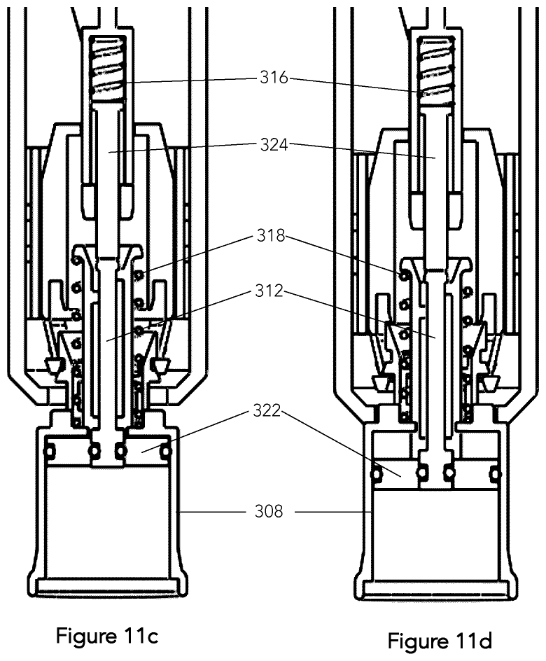

FIG. 11a is a close-up cross-sectional view of a releasable vacuum cap device 320 comprising a vacuum chamber 308. The vacuum cap device 320 can be partially reusable, or preferably made from disposable parts such that the entire vacuum cap device 320 can be a single use device that is ejected from the ISF extraction device and disposed of after each use. When engaged with an ISF extraction device, the second spring 318 in the vacuum cap device 320 is compressed such that it is biased to expand, and the plunger 312 with the plunger seal 322 generally stays at the same level in the vacuum chamber 308 due to the resistance from the compressed second spring 318. The plunger seal 322 is engaged with the plunger 312 in the key-lock structure at the upper region of the plunger 312.

FIG. 11b is a close-up cross-sectional view of the ISF extraction device shown in FIG. 9b with an engaged releasable vacuum cup device comprising vacuum chamber 308. In use, when the vacuum cap device is picked up using the ISF extraction device, the top of the vacuum chamber 308 is engaged with the first stage of a holding mechanism 314 inside the ISF extraction device. Although a housing mechanism is shown here as a tapered movable clip, it is understood that the holding mechanism can be any capable of securely and releasably engaging with the top of a vacuum cap that fulfils the described function. The piston rod 324 of the ISF extraction device which is initially sticking out due to pressure from the upper first spring 316 is pushed up by the plunger 312 of the vacuum cap device, and the upper first spring 316 is compressed in a first engagement position. The vacuum chamber 308 is then placed onto the skin above a site where the skin has been pierced. At this stage, applied pressure from the device housing pressing down onto the skin presses down the vacuum chamber 308 and the top part of vacuum chamber 308 goes up to the second stage or second engagement position in the holder in the device housing while the plunger 312 and plunger seal 322 generally stay at the same level while the upper or first spring 316 is very compressed. The movement of the vacuum chamber 308 and associated plunger 312 provides a vacuum device to establish negative pressure inside the vacuum chamber to release the ISF.

FIG. 11c is a is a close-up cross-sectional view of the ISF extraction device with an engaged vacuum chamber in a first engagement position and FIG. 11d is a is a close-up cross-sectional view of the ISF extraction device shown in FIG. 9b with an engaged vacuum chamber in a second engagement position. In the transition to the second engagement position, part of the air that was previously in the vacuum chamber 308 is squeezed out. The lower second spring 318 has a tendency to expand and is biased in the downward direction to expel air from the vacuum chamber 308. When engaged with skin the negative pressure or vacuum is generated in the area around the vacuum chamber 308 and plunger seal 322 above the pierced skin. When the index trigger on the ISF extraction device is pressed, the holding mechanism in the ISF extraction device pushes down to where the vacuum chamber 308 is engaged with the ISF extraction device, and the ISF extraction device housing and vacuum cap device are detached, leaving the vacuum chamber 308 engaged with the skin with negative pressure remaining inside the vacuum chamber 308 next to the skin. The plunger 312 inside the vacuum chamber is also expelled upwards due to force exerted by the compressed second spring 318, reducing the pressure inside vacuum chamber 308. Once the ISF extraction step is completed, the ISF extraction device is reengaged with the top of the vacuum cap device. In this position, due to the force balance between the first and second springs 316, 318, the plunger 312 and plunger seal 322 on the vacuum cap device return to the position shown in FIG. 11c and the pressure inside the vacuum chamber 308 is raised to near ambient pressure, enabling the vacuum cap device to be removed from skin. Once the vacuum inside the vacuum chamber has been released, the thumb trigger is pressed to push an expander into the top groove of the vacuum cap device expanding at the top of the plunger seal 322 which engages with the plunger 312. The reversible holding mechanism, here shown as a key-lock structure, detaches and releases the plunger 312 from the plunger seal 322. The reversible holding mechanism or key-lock structure detaches and releases the plunger from the holding mechanism. The highly compressed upper or first spring 316 has enough energy to eject the piston rod 324, ejecting the absorbent pad inside the vacuum chamber which has collected the ISF as shown in FIG. 10b. Finally, the vacuum cap device can be released using the same method by actuating the index trigger.

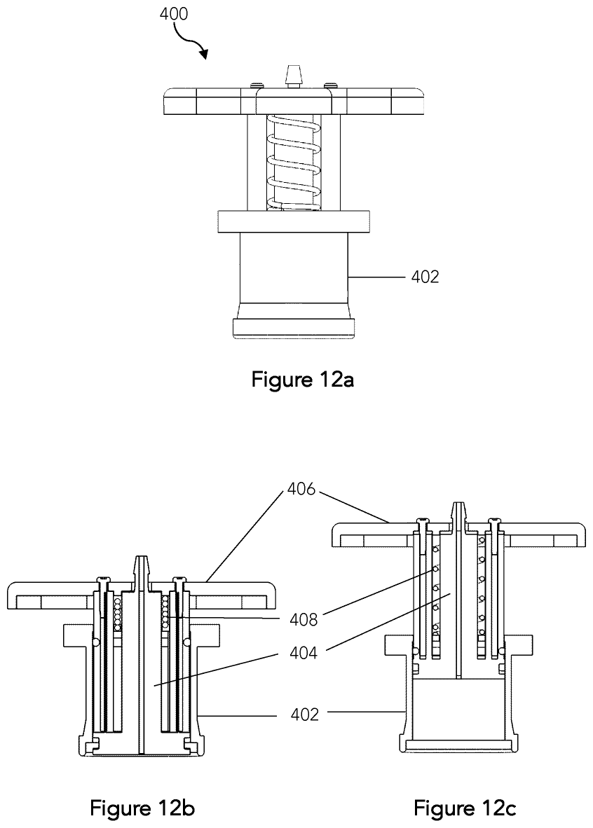

FIG. 12a is a front view of another embodiment of a device for ISF extraction 400 used to create a vacuum over the ISF extraction locus using a vacuum cap 402.

FIG. 12b is a side cross-sectional view of the embodiment of the device for ISF extraction shown in FIG. 12a in a compressed position and FIG. 12c is a side cross-sectional view of the embodiment of the device for ISF extraction in FIG. 12a in an extended position. The device comprises a vacuum cap 402, a plunger 404 with tip inside the vacuum cap 402 and a handle 406 outside the vacuum cap 402, a spring 408 around the plunger rod, and spacer(s) between the vacuum cap and the handle. In use, the handle 406 of the plunger 404 is pressed toward the body of the plunger to move the plunger tip downwards before attaching the device to the skin. The maximum travel distance (and the maximum vacuum level) is determined by the length of the plunger rod. Once pressed down, the vacuum cap 402 on the device is attached to the skin to form an air-tight seal between the vacuum cap 402 and skin. The pressing force on the handle is then released so the compressed spring pulls the plunger tip back, so a vacuum is created. After ISF extraction, the vacuum can be released by tilting the device on the skin to let air enter the cap, or by pressing the handle downwards again to balance the pressure between the ambient air and air inside the cup.

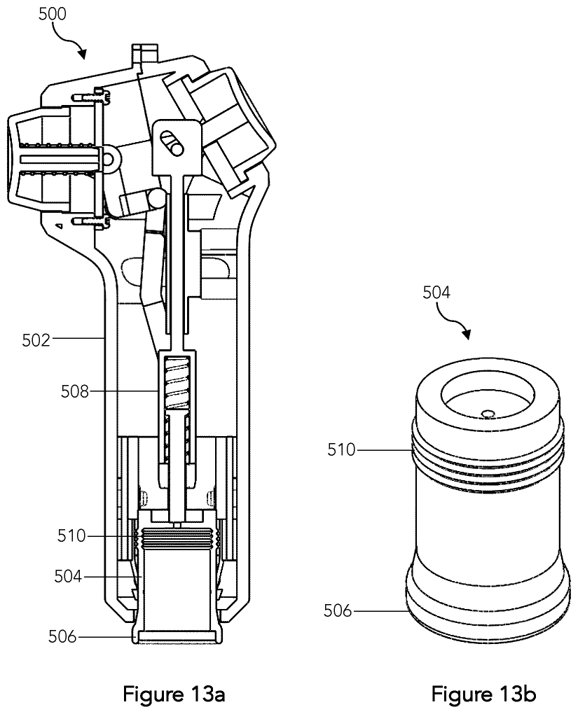

FIG. 13a is a front view of a system for ISF extraction. The system for ISF extraction 500 shown in FIG. 13a is a combination extraction assist device 502 and a vacuum chamber comprising a deformable diaphragm 504 having a rim 506 for contacting a skin surface. The deformable diaphragm 504 can be made of any material such that pressure applied to the diaphragm in a particular way reduces the volume of the diaphragm. Diaphragm 504 is shown with solid sides and top with deformable surface 510 shown as an accordion or compressible spring-like side structure biased in an extended position. Other materials and designs can be used, for example, rubberized materials, or a combination of deformable and hard materials. In use, a gripping mechanism in the extraction assist device 502 acts to releasably grip the diaphragm 504 so that the diaphragm 504 can be engaged and disengaged from the extraction assist device 502 as needed. The gripping mechanism can comprise one or more bore and plug combinations, clips, pressure-assist devices, or any other gripping mechanism known to the skilled person. A pressure mechanism 508 in the extraction assist device 502 is used to apply pressure to the deformable diaphragm 504 to evacuate the air from the diaphragm. Activation of the pressure mechanism 508 to compress the diaphragm 504 can be done either before or after contact with skin, and release of the pressure from the pressure mechanism 508 on the diaphragm 504 once engaged with skin will create a negative pressure environment between diaphragm 504 and the skin surface to assist with withdrawal of ISF from the skin. The pressure mechanism 508 can be a piston as shown, or can be any other mechanism that exerts pressure on the outside of the diaphragm 504 and acts to deform the deformable diaphragm at the deformable surface 510 to reduce the overall volume inside the diaphragm. An actuator to activate the pressure mechanism, on the extraction assist device 502 allows the user to control the deformation of the diaphragm.

An optional absorbent pad (not shown) can be positioned inside the diaphragm 504 such that it is either directly in contact with skin during ISF extraction or positioned just above the skin during extraction. In one embodiment the absorbent pad can be a membrane in contact with some or all of the rim of the diaphragm. In another embodiment the absorbent pad can be secured to a location on the inside of the diaphragm. The absorbent pad can also be engaged with both the inside of the diaphragm and the rim of the diaphragm. It is understood that when ISF is extracted from skin under negative pressure the ISF bubbles or pools above the skin and the absorbent pad is positioned such that it will absorb the ISF. The diaphragm 504 can be removed from the skin surface after ISF extraction by releasing the negative pressure between the diaphragm and the skin surface.

FIG. 13b is a top perspective view of a diaphragm 504 for ISF extraction having an accordion-structure that is deformable under pressure. The diaphragm and optional absorbent pad can be disposable. Depression or pressure on the deformable diaphragm having a deformable surface 510 or semi-flexible deformable surface material when the rim 506 or aperture of the of the diaphragm is in air-tight contact with an surface creates a vacuum or negative pressure inside the diaphragm releasably securing the diaphragm to the surface. Releasing the pressure in the diaphragm will release the diaphragm from the surface.

The following non-limiting examples demonstrate use of the presently described device and method.

Experiment 1—ISF Extraction

Preparation: Before the ISF extraction process, a location on the subject's forearm was identified. Long hairs at this location were shaved and the skin was cleaned using an alcohol disinfecting solution. A sterile microneedle array was attached to the microneedle applicator. The microneedle array microneedle density and microneedle height were varied and recorded.

Skin piercing: The applicator loaded with the microneedle array was pressed onto the skin until the skin domed up and the microneedle array was aligned with the selected location. The loaded spring of the device was then released and triggered the launching of the microneedle array. The microneedle array was driven by the spring force and impacted the subject's skin with a force of 1-2N and a speed of 1-3 m/s. Once the microneedle array landed onto the skin, the applicator remained against the skin for 1-20 seconds before retraction of the applicator from the skin. In order to extract ISF more efficiently, this skin piercing step was repeated at least one more time. In each repetition, the microneedle array impacted the same location on the skin but with the microneedle array rotated to avoid the overlapping of the piercing locations. Typically, the microneedles should generate 1.4-20 piercings per mm². After skin piercing, the applicator was removed and a delay time of 0-10 min was waited before ISF extraction.

ISF extraction: A vacuum with a genitive pressure of 3-9 psi was applied at the pierced skin surface for 5-60 min. The vacuum was built up at a rate of 0.2-6 psi/s. During this course, ISF was extracted and collected on the skin surface in a small bubble or puddle. Once the extraction time was up the vacuum was released and clear ISF was visible at the skin surface.

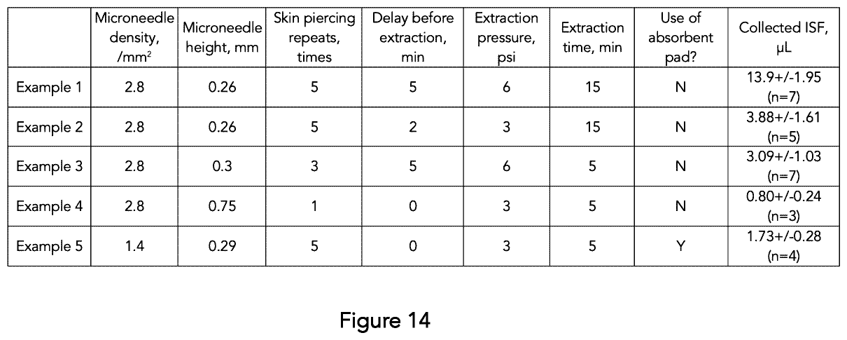

ISF collection: ISF was collected using a glass capillary tube or absorbent pad by dipping one end of the tube or pad to the ISF puddle at the skin surface. The capillary force drives the ISF to the inside of the tubes or pads. The results from Experiment 1 are shown in the table in FIG. 14.

Experiment 2—ISF Extraction Using an Absorbent Pad on the Microneedle Array

Preparation: Before the ISF extraction process, a location on the subject's forearm was identified. Long hairs at this location were shaved and the skin was cleaned using an alcohol disinfecting solution. A sterile microneedle array was attached to the microneedle applicator. No less than one layer of absorbent pad of 80 μm-thick delicate task wipe or other equivalent pads/membranes was attached to needle side of the microneedle array to cover the needle tips. The absorbent pad can also be directly attached to the selected location on the skin using adhesive. The adhesive was only applied at the peripheral of the pad and not in the center of the pad.

Skin piercing: The applicator loaded with a microneedle array was pressed onto the skin until the skin domed up, and the microneedle array was aligned with the selected location. The loaded spring in the ISF extraction device was released which triggered the launching of the microneedle array. The microneedle array was driven toward the skin by the spring force and impact the subject's skin with a force of 1-2N and a speed of 1-3 m/s. Once the microneedle array landed onto the skin, the array was pressed against the skin for 1-20 seconds before the microneedle array was removed from the skin. After this first skin piercing, the absorbent pad was stuck to the skin by another adhesive which was pre-applied onto the pad. In order to extract ISF more efficiently, this skin piercing step was repeated at least one time. In each repetition, the microneedle array impacted the same location on the skin through the pad but with the microneedle array rotated to avoid the overlapping of the piercing locations. Typically, the microneedles generate 1.4-20 microneedle piercings per mm². After skin piercing, the applicator was removed and the was a delay time of 0-10 minutes before ISF extraction.

ISF extraction and collection: A vacuum with a genitive pressure of 3-9 psi was applied at the pierced skin surface over the absorbent pad for 5-60 min. The vacuum was built up at a rate of 0.2-6 psi/s. During vacuum application ISF was drawn up to the skin surface and absorbed by the pad. Once the extraction time was up, the vacuum was released and the absorbent pad with collected ISF was peeled off from the skin.

The results of Examples 1 and 2 are shown in FIG. 14.

Experiment 3—ISF Collection with Multiple Sites

Preparation and Skin Piercing: The subject's forearm was first cleaned using an alcohol disinfecting solution in preparation for a microneedle patch method. A sterile microneedle patch was attached to the microneedle applicator and an ISF extraction device was used to apply the microneedles and rotate the microneedles on the skin for a total of 5 rotation-application cycles. Subjects reported that the process felt as if someone had flicked the skin gently with their finger and was not painful. Three new areas on the subject's forearm was selected the same process was repeated for each new area. There was a waiting period of 5 minutes before proceeding with ISF extraction.

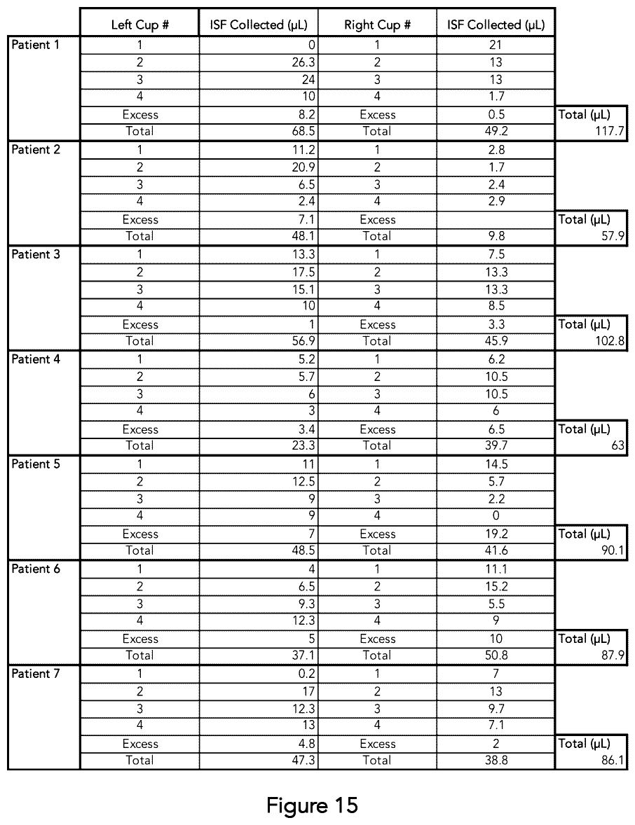

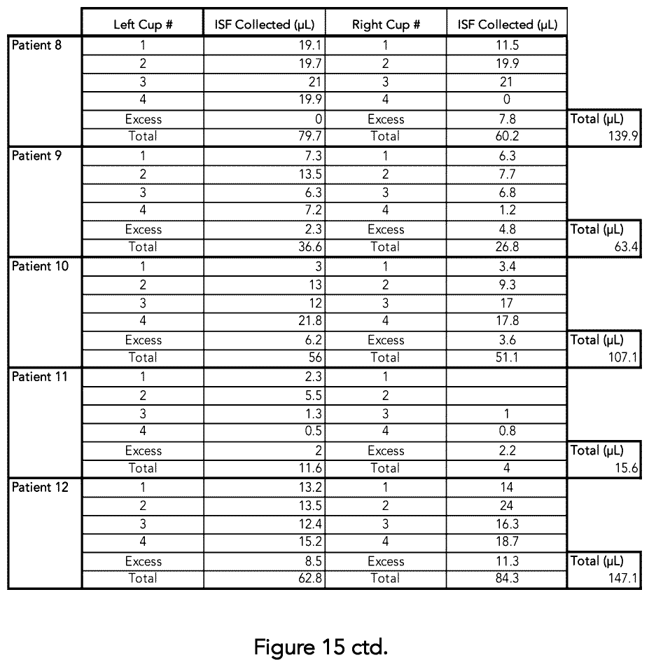

ISF Extraction: A small vacuum device with a vacuum cup was applied to the areas on subject's forearm where the microneedle patch was applied, and the vacuum device was left on the skin for 15 minutes. After 15 minutes, the small vacuum device was removed from the skin, and the ISF skin fluid was collected for analysis. ISF fluid was clearly visible on the skin, collecting in a bubble of fluid above each puncture site. The ISF at each puncture location was collected using a volumetric glass capillary with one end dipped into the ISF puddle. A volumetric glass capillary was also used to collect any ISF that remained on the vacuum cup. FIGS. 15 (and 15 ctd.) is a table showing the results from Experiment 3. Using the described ISF extraction methods and devices, an ISF extraction rate of 9.84/min per user has been achieved. The described efficient ISF extraction methods and devices can be used for medical diagnostics applications.

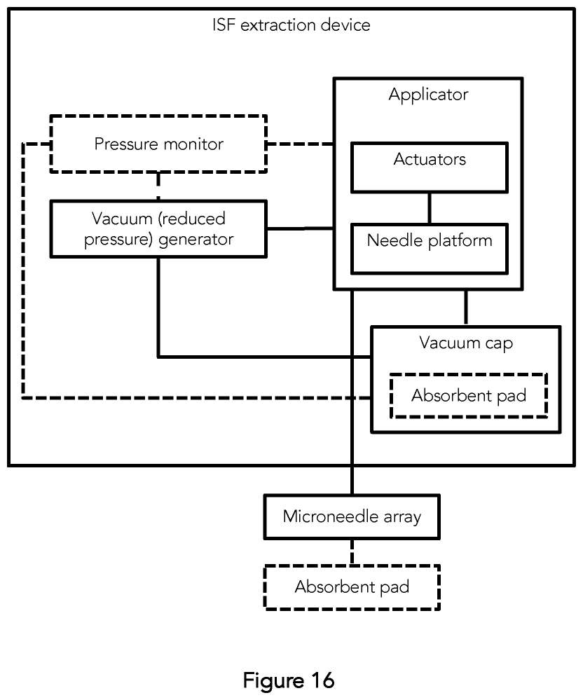

FIG. 16 is a schematic for a general embodiment of an ISF extraction device. The ISF extraction device can have various versions including a vacuum generator combined with a vacuum cap, vacuum cap only, or other additional discrete components and optional features shown in FIG. 16 with dashed lines.

Although omitted for conciseness, the preferred embodiments include every combination and permutation of the various system components and method steps. All publications, patents and patent applications mentioned in this specification are indicative of the level of skill of those skilled in the art to which this invention pertains and are herein incorporated by reference. The invention being thus described, it will be obvious that the same may be varied in many ways. Such variations are not to be regarded as a departure from the scope of the invention, and all such modifications as would be obvious to one skilled in the art are intended to be included within the scope of the following claims.

1. A device for interstitial fluid extraction comprising:
a vacuum chamber having a rim for contacting skin;
a plunger extending through the vacuum chamber, the plunger engaging a piercing end with a microneedle array;
a vertical translation mechanism coupled to the plunger to extend and retract the plunger;
a positioning mechanism coupled to the plunger to position the microneedle array;
a vacuum device for creating a negative pressure inside the vacuum chamber; and
an actuator to activate the vertical translation mechanism and the positional translation mechanism to cause the plunger to execute a piercing cycle of:
extending the piercing end of the plunger toward the rim of the vacuum chamber;
retracting the piercing end of the plunger; and
repositioning the microneedle array.

2. The device of claim 1, wherein the positioning mechanism rotates the microneedle array.

3. The device of claim 1, wherein the positioning mechanism laterally translates the microneedle array.

4. The device of claim 1, wherein the positioning mechanism laterally translates the microneedle array by imprecision in the positional translation mechanism.

5. The device of claim 1, further comprising a vacuum device to apply negative pressure to the vacuum chamber.

6. The device of claim 1, wherein the device is configured to execute the piercing cycle a plurality of times upon activation of the actuator.

7. The device of claim 1, wherein the vertical translation mechanism releases kinetic energy to extend the plunger.

8. The device of claim 1, further comprising an electrical, electro-mechanical, mechanical, or electronic device to control one or more of the plunger, a vertical translation mechanism, positional translation mechanism, vacuum, and actuator.

9. The device of claim 1, further comprising an absorbent pad in the vacuum chamber.

10. A method for extracting interstitial fluid comprising:
executing a piercing cycle of:
contacting a microneedle array at a location on a skin surface;
retracting the microneedle array from the skin surface; and
repositioning the microneedle array to a repositioned location on the skin surface;
repeating the piercing cycle at least one more time; and
applying a negative pressure to the skin surface to extract the interstitial fluid.

11. The method of claim 10, further comprising waiting a specific period of time between a last piercing cycle and applying the vacuum to the skin.

12. The method of claim 11, wherein the specific period of time is more than 2 minutes.

13. The method of claim 10, further comprising analyzing the fluid for the presence of an analyte.

14. The method of claim 10, further comprising repeating the piercing cycle 3, 4, 5, 6, or more than 6 times.

15. The method of claim 10, wherein the microneedle array is rotated at each repositioning step.

16. The method of claim 10, wherein applying the vacuum to the skin further comprises positioning an absorbent pad inside the vacuum chamber for absorbing interstitial fluid while the negative pressure is being applied to the skin.

17. The method of claim 10, wherein applying a vacuum to the skin surface comprises:
positioning a vacuum chamber onto the skin surface;
applying a vacuum to the vacuum chamber using a vacuum generating device to generate negative pressure inside the vacuum chamber; and
releasing the vacuum chamber onto the skin while retaining negative pressure between the vacuum chamber and the skin surface.

18. A device for interstitial fluid extraction comprising:
a diaphragm having a rim for contacting a skin surface; and
an extraction assist device comprising:
a gripping mechanism for releasably gripping the vacuum cap device;
a pressure mechanism to apply pressure to the diaphragm to evacuate the air from the diaphragm; and
an actuator to activate the vertical translation mechanism,
wherein when the diaphragm rim is in contact with the skin surface and the diaphragm is deformed by the pressure mechanism, a vacuum is created in the diaphragm to releasably secure the diaphragm to the skin surface.

19. The device of claim 18, wherein the diaphragm is disposable.

20. The device of claim 18, further comprising an absorbent pad engaged with the diaphragm.

21. The device of claim 20, wherein the absorbent pad is engaged with the inside of the diaphragm, the rim of the diaphragm, or both.
